(19) United States
(12) Patent Application Publication
(10) Pub. No.: US 2005/0171139 A1
(43) Pub. Date: Aug. 4, 2005

(54) Treating psychotic symptoms

(76) Inventors: Ronald P. Hammer JR., Cohasset, MA (US); Kerry E. Culm-Merdek, Natick, MA (US); John J. Byrnes, Ashland, MA (US)

Correspondence Address: FISH & RICHARDSON PC, 225 FRANKLIN ST, BOSTON, MA, 02110, US

(21) Appl. No.: 10/961,101

(22) Filed: Oct. 7, 2004

Related U.S. Application Data
(60) Provisional application No. 60/509,772, filed on Oct. 7, 2003.

(51) Int. Cl. A61K 31/4745; A61K 31/40

(52) U.S. Cl. 514/295; 514/424

(57) ABSTRACT

The invention provides compositions and methods for treating a human diagnosed as having, or at risk for developing, a psychotic symptom by administering a full agonist of a dopamine D2-like receptor to the human. The agonist can be known or identified by screening methods described herein.

### RELATED APPLICATIONS

This application claims the benefit of U.S. Provisional Application No.60/509,772, filed Oct. 7, 2003, which is incorporated herein by reference in its entirety.

### GOVERNMENT SUPPORT

The work described herein was carried out, at least in part, using funds from the U.S. government under grant number MH60251 awarded by the National Institutes of Mental Health. The government may therefore have certain rights in the invention.

### BACKGROUND

Neurotransmitters are chemical substances that mediate the transfer of information between neurons. When an electrical signal travels down the axon of a presynaptic neuron and reaches the axon terminal, neurotransmitters are secreted into the synaptic cleft. Upon traversing the cleft, they bind receptors on the postsynaptic neuron and trigger a series of events that generate an electrical signal in the postsynaptic neuron, thereby perpetuating the transfer of information from one neuron to the next.

There are a variety of transmitters, including acetylcholine, serotonin, glutamate, epinephrine, norepinephrine, histamine, and dopamine. Receptor signaling can be quite complex, and aberrant neurotransmission is believed to underlie psychotic disorders. For example, dopamine is known to bind at least five receptors, which are designated D1-D5. D1 and D5 are referred to as the “D1-like” receptors and D2-D4 are referred to as “D2-like” receptors. Schizophrenic patients have fewer D1 receptors and more D2 receptors than healthy subjects (Sedvall et al., The Lancet, 346:743-749, 1995), and presynaptic dopamine function is known to be upregulated in schizophrenic patients (Hietala, The Lancet, 346:1130-1131, 1995). Certain antipsychotic drugs stimulate D1 regulated pathways, which increases the D1 to D2 activity balance in the brain, but most antipsychotic drugs currently in use target the D2 receptor to block dopamine binding. These treatments exhibit variable potency at the dopamine D2-like receptors and cause extrapyramidal side effects.

### SUMMARY

In accordance with our interest in mental illness, we carried out studies to quantify alterations of G proteins coupled to D2-like receptors after repeated treatment with the full agonist quinpirole. We also determined whether repeated quinpirole administration could increase the extent to which a weak prepulse stimulus attenuates a startle response in mammals. This phenomenon of attenuation is called prepulse inhibition (PPI), and it has been shown to be deficient in patients with schizophrenia (i.e., in schizophrenic patients, the prepulse does not appropriately attenuate a subsequent response to a startling stimulus). Our studies were based, in part, on our belief that not only can D2-like receptor agonists be used as antipsychotic agents, but also that these agonists can be administered in such a way as to provide an improved clinical outcome for patients who experience psychotic symptoms regardless of the precise cause or underlying disease. The outcome may be improved by virtue of an improvement in psychotic symptoms (e.g., less frequent, fewer, or less intense symptoms); improved consequences upon cessation of treatment (e.g., a longer time until relapse); and/or a reduction in a side effect currently Associated with administration of antipsychotic agents (e.g., reduced extrapyramidal symptoms (EPSs)). These benefits may arise from low dosing regimes such as those described further below.

Accordingly, the invention features methods of treating a patient (e.g., a human patient) who has been diagnosed as having a mental illness (e.g., an illness in which the patient experiences a psychotic symptom, such as a hallucination or delusion) or other event that causes psychotic symptoms (e.g., stress can trigger symptoms of schizophrenia in predisposed individuals). The methods can include administering a composition (e.g., a physiologically acceptable composition) that includes a full agonist of a dopamine D2-like receptor (i.e., a full agonist of a D2, D3, and/or D4 receptor (e.g., a D2, D3, or D4 receptor expressed in a human brain)). In some instances, the full agonist can exhibit preferential affinity for the D2 receptor; in others, the full agonist can exhibit preferential affinity for the D3 receptor; and in yet other instances, the full agonist can exhibit preferential affinity for the D4 receptor. While the amount of the composition (and the amount of the full agonist contained therein) may vary, the amount administered will be sufficient to attenuate a sign or symptom of the illness by, for example, reducing its frequency, severity, or duration. Preferably, the dosage is adjusted so that the patient no longer experiences psychotic symptoms, but lesser clinical outcomes are also beneficial and are within the scope of the present invention. Improvement may be observed in a single patient (e.g., in a single patient before and after treatment) or in a group of patients (e.g., an improvement in a treated population relative to an untreated but otherwise comparable population; a single patient's progress may also be compared to that of an untreated population). As noted above, the dosing regimen can also be such that the patient does not experience (or experiences a more tolerable level of) the movement disorders or other side effects that are sometimes associated with antipsychotic agents (e.g., EPSs, which are described further below). A more tolerable level of an EPS may be achieved, for example, when the EPS is less extreme or severe or occurs less frequently or for shorter periods of time. The dosing regimen can also be such that, upon cessation of treatment, the return of one or more of the patient's symptoms is delayed (e.g., the patient may remain symptom free (or in another improved state) for longer than one would expect if an agent other than a full agonist were administered. For example, a patient being treated by regular administration of a low dose of a full D2 agonist may, upon stopping the treatment or disrupting the treatment schedule, remain symptom free (or in whatever improved state they had attained) longer than they would have, had they been treated with an antagonist of a D2-like receptor or if a higher dose of the full D2-like receptor agonist were administered. This benefit would provide time for intervention before a patient experienced a return of a psychotic symptom or became more ill. For example, if a patient, against his doctor's orders, stopped taking his medication (or simply forgot to do so), a family member, friend, or health care professional may notice that fact and help the patient resume the treatment before the patient began to experience psychotic symptoms. Upon cessation of the treatment, the time that passes before a patient experiences a worsened condition may vary. For example, it may be extended by 10, 20, 30% or more beyond what one would expect with a different agent (e.g., a D2-like receptor antagonist) or with the same agent administered at a higher dose. We expect the length of time prior to relapse to vary depending on, for example, the full agonist used and the individual to whom it is administered. While the methods of the invention are not limited to those in which the D2-like receptor agonists impart their benefit by any particular mechanism, we hypothesize that side effects may be lessened due to the specificity of the agonists for cells in the nucleus accumbens and that an acquired tolerance of the receptor may extend the benefits of treatment even after treatment has been stopped or tapered off. Given these benefits, the methods and compositions of the invention may be particularly well suited for long-term treatment of a human diagnosed as having a psychotic symptom, as occurs in the event of neuropsychiatric disorders such as schizophrenia.

In specific embodiments, the patient can receive less than 0.4 mg/kg/day of the full agonist in a single daily dose or in subdivided doses (e.g., a smaller dose taken 2, 3, or 4 times daily), and treatment can continue for days, weeks, months, or years. For example, the patient can receive a full agonist of a D2-like receptor once, twice, three times, or four times per day, and that administration can continue for 2, 3, 4, 5, 6 or more days; 1, 2, 3, 4, 5, 6 or more weeks; 1, 2, 3, 4, 5, 6 or more months; or 1, 2, 3, 4, 5, 6 or more years, with or without interruption (as noted above, a patient may forget or decide not to take their medication on one or more occasions). The dosage of the full agonist can be about 0.001-0.39 mg/kg/day (e.g., about 0.001, 0.005, 0.01, 0,02, 0.03, 0.04, 0.05, 0.10, 0.15, 0.20, 0.25, 0.30, 0.35 or 0.39 mg/kg/day). The methods can involve administration of an initial dose (or series of doses) at a lower concentration and an increase in dosage over time.

While patients amenable to treatment are described further below, we note here that the patient can be a human patient diagnosed as having, or at risk for developing, a psychotic symptom or a condition characterized by one or more psychotic symptoms (e.g., schizophrenia, an obsessive compulsive disorder, depression, a bipolar disorder, or Tourette's syndrome). The psychotic symptoms can include delusions, hallucinations, disorganized speech, grossly disorganized or catatonic behavior, and the like. More generally, the symptoms can include any manifestation that the patient cannot properly process sensory information, has a distorted view of reality, or is otherwise mentally ill and experiencing positive or negative symptoms.

As noted above, the agents administered in the context of the present treatment methods can agonize (e.g., act as a full agonist of) a D2-like receptor (we may refer to agents that act as full agonists of a D2-like receptor as simply “full agonists”). A full agonist can target (by, for example, binding to and activating) a D2, D3, or D4 receptor (or a combination thereof), or spliced or modified variants thereof (e.g., an variant that is-expressed in neurons within the brain (e.g., neurons that express dopamine receptors, such as those within the nucleus accumbens)). Full agonists stimulate dopamine neurotransmission in the brain, and one can determine whether any given agent is a full agonist by its ability to stimulate dopamine neurotransmission. Such studies can be carried out in cell culture (using, for example, dopaminergic cells that are isolated from the brain, part of a cell line, or within brain slices). Examples of presently known full agonists include ropinirole, quinpirole, pramipexole, 7-hydroxy-dipropylamino tetralin (7-OH-DPAT), bromocriptine, cabergoline, apomorphine and pergolide, and any of these agents can be used in the present methods. Full agonists can be further defined as direct or indirect agonists, the former acting directly on a D2-like receptor and the latter affecting the receptor through the activity of at least one other molecule. Preferably, the agonist is selective for a D2-like receptor, and will therefore minimally impact cells that express a D1-like receptor (e.g., D1 or D5).

Full agonists can be incorporated in pharmaceutical compositions as described further below (as the “first” agent), and those compositions may further include a therapeutically effective amount of a second therapeutic agent, such as an antipsychotic or antidepressant (as the “second” agent). The second therapeutic agent can be, for example, an atypical antipsychotic, such as aripiprazole, risperidone, clozapine, olanzapine, quetiapine, or ziprasidone. Further combinations of three or more agents are also within the scope of the invention.

The invention also features methods for identifying a full D2-receptor agonist that includes administering a test compound to a test subject, such as a mammalian subject (e.g., a mouse, rat, or other laboratory animal) and then testing the subject for Prepulse Inhibition (PPI) (e.g., PPI of the acoustic startle response). The goal of the screening assay will be to determine whether the test compound increases the ability of a weak stimulus (i.e., the prepulse) to attenuate a subsequent startle response. As the prepulse is not as effective in subjects with selected mental illnesses (such as those listed above) as it is in healthy subjects, an attenuation of the subsequent startle response indicates that the test compound is effectively converting the subject to a more desirable status. The test compounds (which may be obtained from commercial suppliers of compound libraries (e.g., libraries of small organic or inorganic compounds) or nucleic acid or peptide libraries) can also be tested (or can alternatively be tested) in any other model system for psychoses (e.g., an animal model of schizophrenia). The test compound can be administered (and preferably is administered) more than one time and the test subject tested for PPI on enough occasions to determine that PPI is attenuated in the subject after a certain number of repetitions. For example, the administration and testing steps can be repeated at least two, three, four, six or ten times or more. The test subject can be tested for PPI after every administration of the D2 receptor agonist, or PPI can be tested after only some administrations. The screening method can be carried out using appropriate controls (e.g., administration of an agonist-free carrier; a “vehicle only” control). The amount of the putative full agonist (i.e., the test compound) administered to the subject can be varied and can be low (e.g., less than 0.4 mg/kg/day).

We have conducted studies that established that the full D2 agonist quinpirole effectively stimulated cAMP-dependent protein kinase A (PKA) activity in the nucleus accumbens (NAc) and also increased phosphorylation of the cAMP response element-binding protein (CREB) in that region of the brain. Accordingly, other screening assays of the invention can identify full agonists of D2-like receptors by exposing cells of the NAc, in vivo and/or in tissue culture, to a potential full agonist (i.e., a test compound) and determining whether the test compound stimulates PKA activity or phosphorylation of CREB. PKA activity and CREB phosphorylation can be assessed using methods known in the art (see, e.g., Culm et al., Neuropsychopharmacology 29:1823-1830, 2004).

The materials, methods, and examples are illustrative only and not intended to be limiting. Other features and advantages of the invention will be apparent from the accompanying drawings and description, and from the claims.

### DESCRIPTION OF DRAWINGS

FIG. 1 is a graph illustrating that Prepulse Inhibition (PPI) is attenuated after 10 days during treatment with acute quinpirole. Animals were treated daily for 10 consecutive days with quinpirole (0.0, n=29; 0.05, n=24; 0.1, n=24; or 0.3 mg/kg, n=23). Percent PPI (mean±SEM (Standard Error of the Mean)) was calculated from data obtained using a prepulse 6 decibels (dB) above ambient noise level. * indicates p<0.05, and *** indicates p<0.001 compared to same day PPI in vehicle group. † indicates p<0.05 compared to day 1 PPI within the treatment group.

FIG. 2 is a graph illustrating that full recovery of PPI occurs after a 28 day repeated quinpirole treatment regimen. Quinpirole was administered daily for 28 consecutive days (n=23 in each group). The effect of drug treatment on PPI was assessed on days 1, 7, 14, 21, 25, and 28. Percent PPI (mean±SEM) was calculated from data obtained using a prepulse 6 dB above ambient noise level. * indicates p<0.05 compared to same day PPI in vehicle group. †-indicates p <0.05, and ††—indicates p <0.01 compared to day 1 PPI within the treatment group.

FIG. 3A is a graph illustrating that a 10 day repeated quinpirole treatment has no effect on dopamine-stimulated [³⁵S]GTPγS binding in the nucleus accumbens (NAc) core. Brain sections obtained from animals treated for 10 days with quinpirole (0.0, 0.05 or 0.1 mg/kg) were incubated with [³⁵S]GTPγS in the absence or presence of increasing concentrations of dopamine (1 μM, 10 μM, 100 μM and 1 mM). Autoradiographic data are expressed as percent binding above basal. Values represent mean±SEM; all treatment groups contained 16 animals.

FIG. 3B is a graph illustrating that a 10 day repeated quinpirole treatment has no effect on dopamine-stimulated [³⁵S]GTPγS binding in the NAc shell. Brain sections obtained from animals treated for 10 days with quinpirole (0.0, 0.05 or 0.1 mg/kg) were incubated with [³⁵S]GTPγS in the absence or presence of increasing concentrations of dopamine (1 μM, 10 μM, 100 μand 1 mM). Autoradiographic data are expressed as percent binding above basal. Values represent mean±EM; all treatment groups contained 16 animals.

FIG. 4 is a graph illustrating that a 28 day repeated quinpirole treatment does not affect basal or dopamine-stimulated [³⁵S]GTPγS binding in the NAc. Brain sections obtained from animals treated 28 days with quinpirole (0.0, 0.05, or 0.1 mg/kg) were incubated with [35 S]GTPγS in the absence or presence of dopamine (100 μM). Autoradiographic data are expressed as percent binding above basal. Values represent mean±SEM; treatment groups contained 7-8 animals. Inset: Basal levels of [35S]GTPγS binding. Data are expressed as mean±SEM in units of μCi/g based on ¹⁴C radiostandards.

FIG. 5 is a Western blot showing that NAc G_{iα} protein levels are unaffected by 28 day repeated quinpirole administration. NAc tissue homogenates were prepared from animals treated chronically with quinpirole (0.0, 0.05 or 0.1 mg/kg). Experimental samples (7 μg/lane) were separated by SDS-PAGE along with known amounts of recombinant G_{iα} protein (50, 100 and 200 ng). Proteins were transferred to ImmobilonP membranes and blots were probed with an antibody specific for G_{iα1-3}. Immunoreactive bands were detected by ECL and analyzed using BioRad Quantity One quantification software.

### DETAILED DESCRIPTION

Here, we further describe compositions and methods useful in, for example, treating a patient (e.g., a human patient) diagnosed as having, or at risk for developing, a symptom of psychosis. For example, the patient may experience hallucinations or delusions, or exhibit agitation, impulsiveness, depression, mania, paranoia, hostility, or a similar inappropriate or undesirable behavior. These symptoms are associated with neuropsychiatric disorders such as schizophrenia, depression, and bipolar disorder (relevant symptoms and other conditions are set out further below). While we tend to use the term “patient,” we may also refer to an “individual” or “subject.” Unless a particular meaning is evident from the context in which these terms are used, no distinction is intended.

The methods include administration of a full agonist of a dopamine D2-like receptor (i.e., an agonist of the D2, D3, and/or D4 receptor). The brain normally regulates dopamine neurotransmission by using presynaptic and postsynaptic D2-like receptors to attain a balanced and stable amount of released dopamine. The pre- and postsynaptic receptors work in concert to stimulate dopamine release in areas of the brain where dopamine concentrations are too low and to inhibit release in areas where they are too high (rev. in Vanni et al., Pharm. Ther. 28:251-253, 2003).

The mammalian dopamine receptor family is encoded by at least five distinct genes, two of which encode D1 like receptors, termed D1/D1A and D5/D1B, and three of which encode dopamine D2-like receptors, termed D2, D3, and D4. The genes encoding the D2-like receptors produce numerous functional splice variants and polymorphic forms of these receptors (Gingrich and Caron, Annu. Rev. Neurosci. 16:299-321, 1993; Civelli et al., Annu. Rev. Pharmacol. Toxicol. 32:281-307, 1993; Jarvie and Caron, Adv. Neurol. 60:325-333, 1993). Dopamine D2-like receptors inhibit adenylate cyclase activity in the brain and bind selectively to agonists (e.g., quinpirole) and antagonists (haloperidol, spiperone, emonopride) of numerous structural classes. By contrast, native D1-like receptors are defined by their ability to promote adenylate cyclase activity.

As noted, the compositions and methods described herein are useful for the treatment of neuropsychiatric disorders, such as (but not limited to) schizophrenia, depression, and bipolar disorder. Before describing the methods further, we illustrate some of the patients amenable to treatment. These patients can be readily identified by physicians (e.g., psychiatrists) or other trained professionals working in the field of mental health care. Identifying a patient having, or at risk for developing, a neuropsychiatric disorder or a symptom of psychosis can be included as a step of the present methods. Risk may be assessed based on medical testing (e.g., behavioral testing and/or genetic testing) and family history. Physicians, perhaps in consultation with each other and their patients, can weigh the risk (or probability) of illness and determine whether or not the risk is sufficient to prescribe medication.

Psychosis is a psychiatric classification for a mental state in which the perception of reality is distorted. Patients experiencing a psychotic episode may experience hallucinations (a false sensory perception in the absence of an external stimulus that may occur in any sensory modality (visual, auditory, olfactory, gustatory, tactile, or mixed)), paranoia (an excessive concern about one's own well-being, sometimes suggesting the individual holds persecutory beliefs concerning a threat to themselves or their property), or delusions (a false belief, such as one that is fanciful or derived from deception). Finding of delusions, for example, indicates a pathological condition. While an underlying disease is assumed, delusions are not associated with any particular disease. Although occurring in the context of many pathological states, delusions have particular importance in the diagnosis of schizophrenia. The patient (and, in all embodiments of the invention, the patient can be a human patient) can be suffering from a positive and/or negative symptom of schizophrenia. “Positive” symptoms refer to excessive behavior, including delusions, hallucinations, disorganized speech and disorganized or otherwise bizarre behavior. “Negative” symptoms refer to behaviors that are less than normal, including lack of emotion, avoidance of eye contact, apathy, long lapses in speech or slow speech, speaking in a monotone, and loss of motivation, energy, or feelings of pleasure. Administration of a D2-like receptor agonist according to the methods described herein can relieve both types of symptoms.

Patients amenable to treatment may also exhibit personality changes and/or persistent disorganized thinking (e.g., the patient may have a formal thought disorder). Underlying disturbances to conscious thought can be classified largely by their effects on speech and writing. For example, affected individuals may exhibit pressure of speech (speaking incessantly and quickly), derailment or flight of ideas (switching topic mid-sentence or inappropriately), thought blocking, rhyming or punning or “word salad,” where individual words are intact but speech is incoherent.

The symptoms of psychosis mentioned above are sometimes accompanied by features such as a lack of insight into the unusual or bizarre nature of one's behavior (e.g., anosognosia (a condition in which a person suffering from a brain injury may be unaware of their handicap or in denial of its existence)). Patients may also have difficulty with social interaction and be unable to carry out the activities required for daily living.

Patients diagnosed as having, or at risk for developing, a mental illness such as schizophrenia can also be identified through standard diagnostic procedures. For example, schizophrenia denotes a persistent, often chronic, mental illness that variously affects behavior, thinking, and emotion. Bipolar disorder is recognized as a form of mood disorder characterized by a variation of mood between a phase of manic or hypomanic elation, hyperactivity, and hyperimagination, and a depressive phase of inhibition, slowness to conceive ideas and move, and anxiety or sadness. Together, these symptoms form what is commonly known as manic depression, which has two principal subtypes (bipolar disorder and major depression).

A psychotic individual may be able to perform actions that require a high level of intellectual effort, and individuals with schizophrenia can have long periods without psychosis. Similarly, patients with bipolar disorder and depression can have mood symptoms without psychosis. Conversely, psychosis can occur in patients without chronic mental illness, such as a result of extreme stress.

The composition administered to the patient (e.g., a patient identified as having one or more of the symptoms described above or a patient at risk thereof) can be a full agonist of a D2-like receptor. Dopamine agonists have been used in the treatment of Parkinson's disease. Accordingly, the present methods represent a new use of dopamine agonists and can be administered not only to patients who exhibit a symptom of psychosis, but also to patients who exhibit such symptoms without suffering from Parkinson's disease.

Examples of dopamine D2-like receptor agonists include ropinirole (Requip™), quinpirole, pramipexole (Mirapex™), 7-hydroxy-dipropylamino tetralin (7-OH-DPAT), bromocriptine (Parlodel™), cabergoline, apomorphine or pergolide (Permax™). In one embodiment, the agonist is selective for a D2 receptor. Other agonists, including those identified by screening libraries (e.g., screening compound libraries to identify compounds that bind (e.g., selectively bind and activate) a D2-like receptor)) can also be used in the present methods. Subsequent to receptor binding, the compositions may (but do not necessarily) function by a mechanism similar to that of known dopamine D2-like receptor agonists. For example, a compound identified in a screen for D2-like agonists may inhibit the stimulation of adenylate cyclase in the brain (e.g., the compound may modify the function of an adenylate cyclase enzyme) or modify protein kinase A (PKA) expression or function. Unless specifically noted, there is no intended distinction between the term “agent” and the term “compound.” Screening methods to identify new agents are described further below.

The first dose of the full agonist and the doses that follow can be relatively low. We provide guidance regarding dosage here both in terms of absolute amounts and in terms of the effect of the agonist. The primary goal of the dosing regime is to provide effective relief of psychotic symptoms while minimizing side effects. Physicians and others can assess the patient's mental health through behavioral tests, conversation, and other observations (e.g., observing the patient's interactions with others). Alternatively, or in addition, the effectiveness of the treatment can be assessed based on the patient's own reports of increased well-being. The treatment can inhibit a psychotic symptom by reducing its frequency, severity, or duration, and we may use the term “inhibit” interchangeably with terms such as “lessen,” “reduce,” “attenuate,” and “improve.” Preferably, the dosage is such that the patient no longer experiences psychotic symptoms or only rarely experiences psychotic symptoms, but lesser clinical outcomes representing any degree of improvement are also beneficial and are within the scope of the present invention. While the first objective of treatment is to improve the patient's psychotic symptoms, the dosage is preferably also one that prevents or attenuates external effects of the full agonist. For example, the first dose of the D2-like receptor agonist and preferably the doses that follow can be low enough to prevent or attenuate any feeling of euphoria, increased energy, or other symptoms that can be described as a “high.” Other adverse reactions or effects resulting from administration of excessive amounts of the full agonist include nausea, headache, muscle soreness, and dizziness. One of ordinary skill in the art will be able to recognize other undesirable side effects caused by drug overdose.

The dosage of the full agonist is also preferably low enough that the patient experiences few (or no) EPSs. EPSs include akinesia (lack of movement or Parkinson-like movement), dystonic reactions (e.g., muscle spasms within the face, neck, or back), dyskinesia (evidenced by, for example, inappropriate blinking or twitches), and/or akathesia (an inability to sit still). EPSs may also be evident as pacing or a total (or almost total) inability to sit still (if forced to sit still, the patient will experience extreme anxiety or agitation). EPSs meeting these criteria may be referred to as akathisia, and they are dangerous because an extremely unpleasant experience is coupled with an intense urge to act (this can lead to suicide or suicidal tendencies). The EPSs may also be evident as involuntary muscle contractions that force specific parts of the body into abnormal movements or positions, sometimes causing pain. Some of the muscular spasms may be associated with particular disorders. For example, muscular spasms of the neck may be referred to as torticollis.

Exemplary daily doses (for a human patient of average weight and without extenuating medical circumstances) can be less than or about 1-40 mg of the agonist (e.g., less than, or about, 40.0, 35.0, 30.0, 25.0, 20.0, 15.0, 10.0, 9.0, 8.0, 7.0, 6.0, 5.0, 4.5, 4.0, 3.5, 3.0, 2.5, 2.0, 1.5, 1.0, 0.5, 0.25, 0.1, 0.075, 0.05, 0.025, or 0.01 mg). As noted above, dosage can also be expressed as an amount of the full agonist per unit of the patient's weight (e.g., mg/kg). For example, in specific embodiments, the patient can receive about 0.4 mg/kg of the agonist. This dosage can be administered daily or several times daily, and treatment can continue for days, weeks, months, or years. We know that high doses of D2 agonists can cause psychotic symptoms; clearly, therapeutic doses must be lower than these doses. The dosage can remain constant over the course of the treatment regimen. In some embodiments, the amount of the agonist administered and the frequency with which it is administered are such that a certain low level of the agonist (e.g., the level of circulating agonist) is maintained. Because the dosing regime (e.g., a low dose, administered chronically) can tolerize cells (e.g., D2 receptor-expressing cells in the brain), making them less responsive to dopamine, the patient is less likely to experience an ill effect (e.g., the return of a positive symptom) if the dosing regime is interrupted (e.g., where the patient forgets or decides not to take their medication). Accordingly, the methods of the invention encompass those for tolerizing a D2-like receptor. Tolerance can be achieved by repeatedly administering low doses of the full agonist. Where a patient has developed a tolerance, a higher dose of the agonist can be administered without causing an adverse reaction (or without causing an adverse reaction that is as significant as it would have been had the patient's treatment been initiated at the higher dosage). Thus, regardless of the initial treatment regime, dosages can be adjusted. As in other therapies, a patient and his or her physician can work together toward an optimal personalized dose. Adjustment (e.g., dosage increase or decrease over time) is contemplated and will preferably be such that the patient experiences an improvement in their psychotic symptoms but does not experience an external effect of the drug or significant EPSs. The low dosages stated above notwithstanding, full agonists of a D2-like receptor can be administered at higher dosages (e.g., by increasing dosage over time).

As with other pharmaceutical agents, data obtained from in vitro or cell culture assays or from animal studies can be used to formulate a range of dosage for use in humans. For example, dosage can be determined in an animal model of a neuropsychiatric disorder (e.g., an animal model of schizophrenia). Such models are known in the art (see, e.g., Kilts, Biol. Psychiatry 50:845-855, 2001 (erratum Biol. Psychiatry 51:346, 2002). We expect the determined dosage to lie within a range of circulating concentrations that include the ED₅₀ with little or no toxicity. Moreover, the dosage may vary within this range depending upon the dosage form employed and the route of administration utilized. Plasma levels of a given full agonist can be measured, for example, by high performance liquid chromatography.

A pharmaceutical composition (which we may also refer to as a therapeutic composition or a physiologically acceptable composition) can include a therapeutically effective amount of an agonist of a dopamine D2-like receptor, including those described herein, formulated for delivery to a patient. The formulations can vary and include those presently used to deliver therapeutic agents to mentally ill patients. For example, pharmaceutical compositions containing a full agonist of a D2-like receptor can be formulated in a conventional manner using one or more physiologically acceptable carriers (which we may also refer to as excipients diluents). Thus, a full agonist (or combinations thereof (e.g., two agonists)) can be formulated for administration by oral or parenteral administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. While we expect most formulations will contain a dosage appropriate for direct administration to a patient, the invention also encompasses compositions in which the full agonist(s) is/are concentrated. Such compositions can be diluted by the patient or, preferably, by a pharmacist or caregiver prior to administration. Liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound. For buccal administration, the compositions may take the form of tablets or lozenges formulated in a conventional manner.

For administration by inhalation, full agonists can be delivered in the form of an aerosol spray presented from pressurized packs or a nebulizer, with the use of a suitable propellant, for example, dichlorodifluoromethane, trichlorofluoromethane, dichlorotetra-fluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator may be formulated containing a powder mix of the composition and a suitable powder base such as lactose or starch.

The pharmaceutical compositions may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

In addition to the formulations described previously, the compositions may also be formulated as a depot preparation. Such long acting formulations may be administered by implantation (for example subcutaneously or intramuscularly) or by intramuscular injection. Thus, for example, full agonists can be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack or dispenser can include metal or plastic fashioned as a blister pack. As is true for any of the formulations described herein, the pack or dispenser device can be packaged and accompanied by instructions for use and, optionally, paraphernalia for administration (e.g., should the formulation be an aerosol, a dispenser can be included).

Specific excipients that can be used in the agonist-containing compositions include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol.

Specific routes of parenteral administration include intravenous, subcutaneous, intramuscular, intracranial, intraorbital, opthalmic, intraventricular, intracapsular, intraspinal, intracisternal, intraperitoneal, transdermal, and transmucosal. As noted above, oral administration is also possible and is favored for its convenience. Methods for making formulations that can be administered by these routes are well known and can be found in, for example, “Remington's Pharmaceutical Sciences.”

The agonist-containing compositions can further include at least a second therapeutic agent, such as an antipsychotic or antidepressant compound. The antipsychotic can be, for example, an atypical antipsychotic, such as aripiprazole, risperidone, clozapine, olanzapine, quetiapine, or ziprasidone; or a typical antipsychotic such as chlorpromazine, fluphenazine, haloperidol, thiothixene, trifluoperazine, perphenazine, or thioridazine. Although these agents can be formulated together with a full agonist of a D2-like receptor, the invention also features methods of treating a patient who has a psychotic symptom by administering the agonist together with second antipsychotic agent (including, but not limited to, those described above). The two agents can be administered by the same or different routes and/or at the same (or essentially the same) time. For example, the patient can be treated by sequential administration of two compositions formulated for oral administration (one containing a full agonist and one containing a distinct antipsychotic agent). Where the first and second agents are formulated in separate compositions, the two compositions can be labeled for use together. For example, the composition including the full D2-like receptor agonist can be administered before, at the same time, or after the composition containing the second therapeutic agent, and the two compositions can be labeled individually or collectively for use in such a manner. The two compositions can be packaged separately or together.

Also provided are methods for identifying a D2-like receptor agonist for use in treating a patient who experiences a psychotic symptom or who has been diagnosed as having a disease or disorder characterized by a psychotic symptom. An exemplary screening method includes administering a test compound to a test subject, such as a mammalian subject (e.g., a mouse or rat) and then assessing (e.g., measuring) the prepulse inhibition of the acoustic startle response (referred to herein as PPI) of the test subject (Swerdlow et al., J. Pharmacol. Exp. Ther: 256:530-536, 1991). As noted above, PPI is the reduction in response when a startling pulse stimulus is preceded by a weak prepulse stimulus (see also the Examples below). The test compound can be administered more than twice, and the test subject tested for PPI on one occasion and, if necessary, on subsequent occasions to determine that PPI is attenuated in the subject. For example, the test compound can be administered to the test subject at least one time daily for at least 2 days, 3 days, 4 days, 6 days, 10 days, 20 days, 30 days, or longer. The test subject or a comparable test subject can be tested for PPI, for example, before the administration of the test compound, and at least 2 times, 3 times, 4 times, 6 times, 10 times or more for PPI. For example, the test subject can be tested for PPI at least once a day following the administration of the test compound (e.g., a test compound administered daily), after every other administration of the compound, or at irregular intervals following the administration of the test compound (for example, after the 1^{st}, 3^{rd}, 5^{th}, 9^{th}, and 20^{th} administrations). The test compound is preferably administered at regular intervals, such as once every day, once every other day, once every third day, etc. In another example, the test compound can be administered twice every day, twice every other day, twice every third day, etc.

A test compound identified as being capable of attenuating PPI can be assessed further as a treatment of a psychotic symptom, disease, or disorder, as described herein (i.e., it can be developed through clinical trials), formulated as a pharmaceutical composition and administered to a patient as described herein.

The invention is further illustrated by the following examples, which do not limit the invention. The contents of all references, pending patent applications and published patents, cited throughout this application are hereby expressly incorporated by reference.

### EXAMPLES

### Example 1

### Experimental Paradigm: PP1 Recovery After Chronic D2 Agonist Treatment

The aberrant neurotransmitter systems that underlie psychotic disorders can be studied in animal models of sensorimotor gating. Sensorimotor gating is defined as the neural process responsible for the integration and processing of sensory information, and it is deficient in patients with schizophrenia (Braff et al., Psychophysiology 15:339-343, 1978). This phenotype can be quantitatively assessed in various species by measuring prepulse inhibition of the acoustic startle response (referred to herein as “Prepulse Inhibition,” or “PPI”) (Swerdlow et al., J. Pharmacol. Exp. Ther. 256:530-536, 1991), which is the reduction in response when a startling pulse stimulus is preceded by a weak prepulse stimulus.

The neural substrate for PPI modulation includes the mesocorticolimbic dopamine system (Swerdlow and Geyer, Schizophr. Bull. 24:285-301, 1998; Swerdlow et al., J. Neurosci. 20:4325-4336, 2001), especially D2-like dopamine receptors in the nucleus accumbens (NAc). When administered systemically, both direct and indirect dopamine agonists disrupt PPI in experimental animals (Mansbach et al., Psychopharmacology (Berl.) 94:507-514, 1988), as do selective D2-like receptor agonists such as quinpirole and 7-hydroxy-dipropylaminotetralin (7-OH-DPAT) (Peng et al., Neuropsychopharmacology 3:211-218, 1990; Varty and Higgins, Behav. Pharmacol. 9:445-455, 1998). The involvement of NAc D2-like receptors in PPI regulation was confirmed by site-specific infusion of quinpirole into the NAc, which elicited PPI disruption (Wan and Swerdlow, Psychopharmacology (Berl.) 113:103-109, 1993).

While the effect of acute dopamine agonist treatment on PPI is well-defined, the effect of chronic drug treatment is less clear. Therefore, we sought to examine PPI adaptation after repeated treatment with the selective D2-like receptor agonist, quinpirole. In addition, we sought to characterize the substrates underlying PPI adaptation following repeated quinpirole treatment by assessing putative changes in D2-like receptor-G protein function. NAc D2-like receptors are coupled to Gᵢ and Gₒ proteins (Stoof and Kebabian, Nature 294:366-368, 1981), which are critical for PPI modulation (Culm et al., Brain Res. 982:12-18, 2003). Chronic cocaine treatment, which attenuates PPI disruption (Byrnes and Hammer, Neuropsychopharmacology 22:551-554, 2000), decreases pertussis toxin-mediated ADP-ribosylation of G_{iα} and G_{oα} and reduces G_{iα} and G_{oα} immunoreactivity in the NAc (Nestler et al., J. Neurochem. 55:1079-1082, 1990). In contrast, repeated treatment with the dopamine antagonist haloperidol increases D2-like receptor binding and efficacy of D2-like receptor-G protein coupling as assessed using [³⁵S]GTPγS binding (Geurts et al., Eur. J. Pharmacol. 382:119-127, 1999), without affecting G_{iα} or G_{oα} levels (Meller and Bohmaker, Neuropharmacology 35:1785-1791, 1996). Therefore, we utilized dopamine-stimulated [³⁵ ]GTPγS binding and immunoblots to quantify putative alterations of G proteins coupled to the D2-like receptor after repeated quinpirole treatment.

### Example 2

### Prepulse Inhibition Testing

Male Sprague-Dawley rats (Charles River Laboratories; Frederick, Md.) weighing 250-300 g were habituated to handling and treatment by placement into a Startle Monitor Behavioral Testing chamber (Hamilton Kinder, Poway, Calif.) with 70 dB ambient noise for five minutes daily on each of two days prior to testing. Animals were treated once daily for 10 consecutive days with the same dose of quinpirole (0.0, 0.05, 0.1 or 0.3 mg/kg sc; RBI-Sigma, Natick, Mass.) in 0.9% sterile saline.

Startle amplitude was determined using the Startle Monitor Behavioral Testing System at baseline and after quinpirole treatment on days 1, 4, 7, and 10. Mean startle amplitude was measured over 150 ms following the presentation of the pulse stimulus in units of Newtons. For baseline testing, each animal was exposed to 70 dB ambient noise for 5 min followed by a test session consisting of the randomized presentation of 32 trials: 17 pulse trials (40 ms 120 dB pulse) and 15 prepulse trials (5 each at 73, 76, and 82 dB with a 20 ms prepulse given 100 ms prior to a 40 ms 120 dB pulse). The mean acoustic startle response to pulse alone trials was used to normalize animals into various treatment groups. Starting 2-3 days later, the first drug challenge test session began 10 minutes after quinpirole treatment and consisted of the randomized presentation of 54 trials: 24 pulse trials, 10 prepulse trials at 73 dB, 10 prepulse trials at 76 dB and 10 prepulse trials at 82 dB. The average inter-trial interval was 15 seconds and percent PPI was calculated using the following equation: 100-[(mean prepulse response/mean pulse response)×100]; a higher percent PPI implies greater inhibition of startle response due to presentation of the prepulse. Percent PPI data calculated for each prepulse level and mean pulse response data on the first and last day of testing were analyzed by separate analyses of variance (ANOVAs) with drug treatment as a between subject factor. Within each treatment group, percent PPI data from days 1, 4, 7 and 10 were analyzed using ANOVA with repeated measures. Post hoc comparisons were conducted using a Dunnett's test. Alpha was 0.05.

### Example 3

### [³⁵S]GTPγS Binding Analysis

Immediately after PPI testing on day 10, animals were decapitated and brains were removed, rapidly frozen at −30° C. in 2-methylbutane, and sectioned at 16 mm using a −20° C. cryostat. Sections corresponding to approximately 1.7 mm anterior to bregma were collected, thaw mounted onto gelatin-coated slides, and stored at −80° C. for a maximum of seven days prior to the time of the binding assay. Animals from the high dose group were not assessed because no behavioral adaptations occurred with repeated treatment.

[³⁵S]GTPγS binding was performed as described previously (He et al., Brain. Res. 885:133-136, 2000). Briefly, sections were preincubated in assay buffer (50 mM Tris-HCl, 3 mM MgCl₂, 0.2 mM EGTA, and 100 mM NaCl, pH 7.4) for 15 minutes at 25° C. followed by a 15 min incubation in the same buffer supplemented with 2 mM GDP (ICN, Costa Mesa, Calif.). Sections were then incubated in assay buffer containing 2 MM GDP and 50 pM [35 S]GTPγS (NEN-Perkin Elmer Life Sciences, Boston, Mass.) in the absence (basal) or presence of 1 μM, 10 μM, 100 μM or 1 mM dopamine (Sigma-Aldrich, St. Louis, Mo.) for 1 hour at 25° C. The sections were washed two times for 3 minutes at 4° C. in 50 mM Tris-HCl (pH 7.4) and briefly rinsed in distilled water. After air drying, slides were co-exposed with ¹⁴C radiostandards (ARC-146; American Radiolabeled Chemicals, St. Louis, Mo.) to x-ray film (Biomax MR, Eastman Kodak Company, Rochester, N.Y.) for 4 days. Autoradiographic images were analyzed using NIH Image (developed by Wayne Rasband, National Institutes of Health) to determine the relative amount of ligand binding using a calibration curve constructed in terms of μCi/g based on the ¹⁴C radiostandards. Data were expressed as percent binding above basal levels. Nonlinear regression analysis was used to calculate maximal efficacy and log EC50 values from sigmoidal dose-response binding curves using GraphPad™ Prism (GraphPad Software, San Diego, Calif.). Maximal efficacy and log EC50 values were compared using a one-way analysis of variance. Log EC50 data are expressed in terms of the EC50 and its 95% confidence interval (CI) for each treatment group (the CI is symmetrical on a log scale, but asymmetrical when converted to EC50).

### Example 4

### Western Blot Analysis of G_{iα} and G_{sα} protein.

Male Sprague-Dawley rats (Charles River Laboratories, Kingston, R.I.) weighing 250-300 g were habituated to handling and placed in the behavioral test chambers for two days. Animals were treated once daily for 28 consecutive days with the same dose of quinpirole (0.0, 0.05, or 0.1 mg/kg). Treatment groups were normalized according to the mean acoustic startle response observed during baseline testing. The effect of chronic drug exposure on PPI was assessed on days 1, 7, 14, 21, 25 and 28. Percent PPI data calculated for each prepulse level and mean pulse response data on the first and last day of testing were analyzed by separate ANOVAs with drug treatment as a between subject factor. Within each treatment group, PPI data from days 1, 7, 14, 21, 25, and 28 were analyzed by ANOVA with repeated measures. Post hoc comparisons were conducted using a Dunnett's test. Following PPI testing on day 28, animals were decapitated, their brains were removed and frozen at −30° C. in 2-methylbutane. A subset of brains from each treatment group were sectioned using a −20° C. cryostat to a level corresponding to 1.7 mm anterior to bregma; the remaining brains were utilized for [³⁵S]GTPγS binding analysis (described below). A unilateral 2 mm wide micropunch (1 mm deep) of the NAc was obtained and homogenized in ice-cold homogenization buffer (50 mM Tris, pH 7.4, 1 mM dithiothreitol, 1 mM EGTA, 10 μg/ml leupeptin, 20 μg/ml aprotinin). Homogenized tissue was centrifuged at 10,000 g and the resulting pellet was resuspended in 100 ml of homogenization buffer. Protein content in tissue homogenates was calculated using a protein assay kit (Bio-Rad, Hercules, Calif.), and the samples were stored at −80° C. prior to analysis.

Aliquots of tissue homogenates were separated by SDS-PAGE using 12% polyacrylamide gels. Seven and fourteen jg of total protein were loaded per lane for G_{iα} and G_{sα} respectively. All experimental samples were analyzed in duplicate. Recombinant G_{iα1} or G_{sα} protein (Santa Cruz Biotechnology, Santa Cruz, Calif.) was used as calibration standards on each gel at concentrations of 50, 100 and 200 ng/well for G_{iα} and 40, 80 and 160 ng/well for G_{sα}. The recombinant G_{iα} protein utilized migrates with a molecular weight of 42 kDa, approximately 1 kDa larger than endogenous G_{iα} due to the addition of an epitope tag.

After gel electrophoresis, proteins were transferred to membranes (Immobilon-P™; Millipore, Bedford, Mass.) then incubated overnight at 4° C. in 5% blocking solution (5% dry milk, 1×TBS/0.05% Tween). Membranes were incubated for one hour at room temperature in antisera specific for either the carboxy terminus of G_{iα1-3} of rat origin (1:500 dilution, C-10, Santa Cruz Biotechnology, Inc.; Santa Cruz, Calif.) or the carboxy terminus of G_{sα} of rat origin (1:400 dilution, C-18; Santa Cruz Biotechnology, Inc.; Santa Cruz, Calif.). Blots were incubated for one hour at room temperature in a 1: 10,000 dilution of HRP-conjugated donkey anti-rabbit IgG (Pierce Biotechnology; Rockford, Ill.) in 1% blocking solution, and washed twice in 1×TBS/0.05% Tween and once in 1×TBS. Immunolabeled bands were detected using chemiluminescence (ECL Plus; Amersham Biosciences; Piscataway, N.J.). Quantification software (Quantity One, Bio-Rad Laboratories; Hercules, Calif.) was utilized to generate a standard curve of density X pixel area versus ng of recombinant G_{α} protein. These standard curves were used to calculate the relative immunoreactivity of bands representing G_{iα} with a molecular weight of 40-41 kDa or G_{sα} with a molecular weight of 42 and 47 kDa (Self et al., J. Neurosci. 14:6239-6247, 1994). Data were compared using a one-way ANOVA.

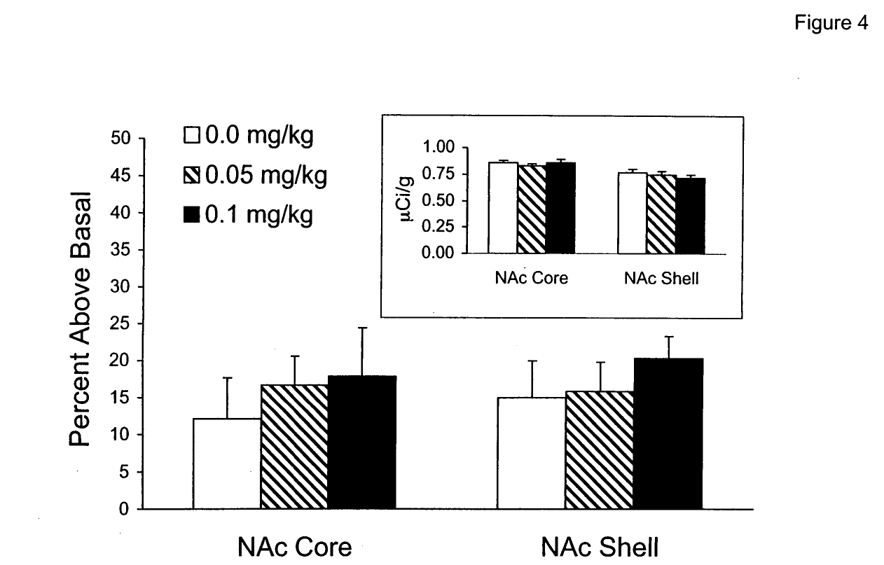

Immediately after PPI testing on day 28, animals were decapitated, and brain tissue was processed for [³⁵S]GTPγS binding analysis as described above. Following preincubation, the sections were incubated in assay buffer containing 2 mM GDP (ICN, Costa Mesa, Calif.) and 50 pM [³⁵S]GTPγS (NEN/Perkin Elmer Life Sciences, Boston, Mass.) in the absence (basal) or presence of 100 μM dopamine (Sigma-Aldrich, St. Louis, Mo.) as described above. Only one concentration of dopamine (100 μM), which elicits maximal [³⁵S]GTPιS binding (He et al., Brain Res. 885:133-136, 2000), was utilized in this assay because previous results indicated that repeated quinpirole treatment did not affect maximal efficacy or EC50 values. After sections were washed, dried and exposed to x-ray film, the relative amount of ligand binding was determined using a calibration curve based on ¹⁴C radiostandards co-exposed to film. Data were expressed as percent binding above basal (FIG. 4). The effect of chronic quinpirole administration on basal and dopamine-stimulated [³⁵S]GTPγS binding was assessed using one-way ANOVA.

Throughout the study, animals were provided with food and water ad libitum while housed in a climate-controlled facility with 12-hour reverse light/dark cycles (lights off at 0900 h). Animals were allowed to acclimate to the laboratory for seven days prior to handling. All experiments were approved by the Tufts-New England Medical Center Animal Care and Use Committee and conducted in accordance with the National Institute of Health Guide for the Care and Use of Laboratory Animals.

### Example 5

### Repeated Treatments Attenuate D2-like Agonist-Induced PPI Disruption

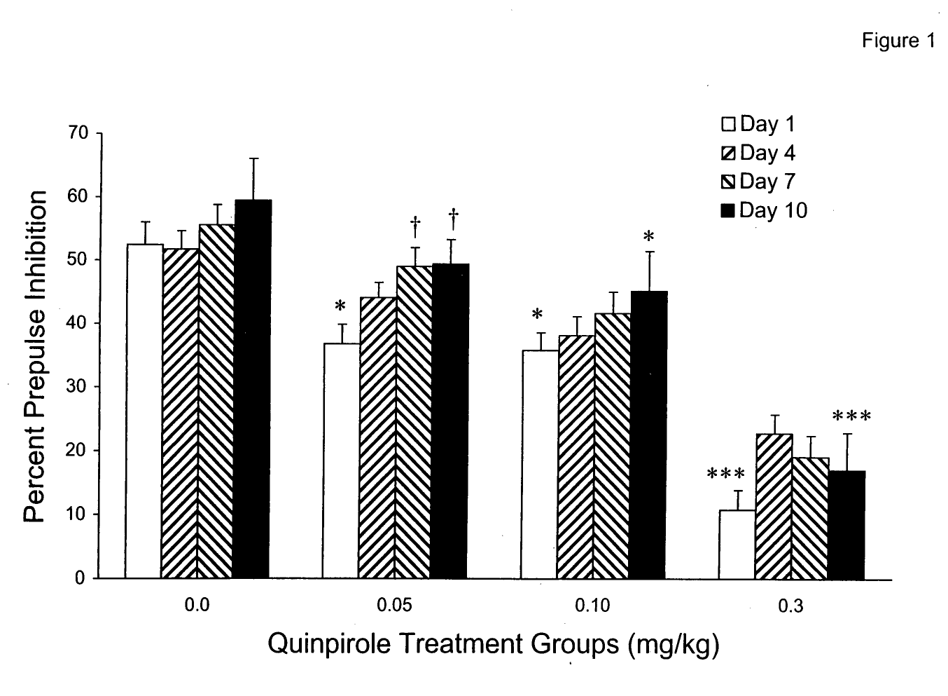

Acute quinpirole treatment significantly reduced PPI at all doses examined. Increasing quinpirole doses reduced PPI by 30%, 31 % and 79% compared to vehicle treatment following a 76 dB prepulse [F(3,93)=16.7, p ≦0.005] (FIG. 1). Repeated administration of the lowest dose (0.05 mg/kg) attenuated the ability of quinpirole to reduce PPI. By day 10, PPI had increased significantly by 34% and 38% compared to day 1 following 76 dB and 82 dB prepulses, respectively [76 dB: F(3,93)=3.4, p‘0.05; 82 dB: F(3,92)=8.6, p≦0.005] (FIG. 1 and Table 1).

TABLE 1
Effect of acute and 10 day repeated quinpirole administration on percent PPI
following a 73 or 82 dB prepulse.

| | Quinpirole Dose (mg/kg): | | | | | | | |
|---|---|---|---|---|---|---|---|---|
| Prepulse | 0.0 | 0.0 | 0.05 | 0.05 | 0.1 | 0.1 | 0.3 | 0.3 |
| Level | Day 1 | Day 10 | Day 1 | Day 10 | Day 1 | Day 10 | Day 1 | Day 10 |
| 73 dB | 35.8 ± 4.0 | 40.8 ± 5.3 | 20.8 ± 3.1^{a} | 31.9 ± 2.9^{b} | 17.9 ± 3.4^{a} | 20.4 ± 4^{a} | −1.0 ± 5.4^{a} | 1.0 ± 5.0^{a} |
| 76 dB | 52.4 ± 3.6 | 59.5 ± 3.1 | 36.9 ± 2.9^{a} | 49.5 ± 3.0^{b} | 36.0 ± 3.2^{a} | 45.4 ± 3.3^{a} | 10.8 ± 6.6^{a} | 17.2 ± 5.8^{a} |
| 82 dB | 66.9 ± 2.7 | 70.6 ± 3.7 | 46.3 ± 2.7^{a} | 63.9 ± 1.9^{b} | 46.6 ± 3.4^{a} | 60.6 ± 3.0^{b} | 34.4 ± 5.0^{a} | 41.4 ± 5.4^{a} |

Data are expressed in terms of percent PPI ± SEM (Standard Error of the Mean).
^{a}indicates p ≦ 0.05 compared to same day PPI in vehicle group.
^{b}indicates p ≦ 0.05 compared to day 1 PPI within the treatment group.

PPI increased slightly upon repeated treatment with the intermediate dose (0.1 mg/kg), but this change was significant only following 82 dB prepulses [F(3,92)=4.8, p≦0.005] (Table 1). Repeated treatment with the highest dose (0.3 mg/kg) did not affect PPI disruption following any prepulse level. Neither acute nor repeated quinpirole treatment altered mean startle responses to pulse trials in the low or intermediate dose groups. However, mean startle response was significantly reduced on day 10 in the high dose group (Table 2).

TABLE 2
Effect of 10 day repeated quinpirole administration on mean acoustic
startle response.

| | Quinpirole Dose (mg/kg): | | | |
|---|---|---|---|---|
| | 0.0 | 0.05 | 0.1 | 0.3 |
| Day 1 | 0.44 ± 0.05 | 0.33 ± 0.06 | 0.39 ± 0.04 | 0.31 ± 0.04 |
| Day 4 | 0.45 ± 0.05 | 0.40 ± 0.04 | 0.42 ± 0.04 | 0.29 ± 0.04 |
| Day 7 | 0.40 ± 0.05 | 0.38 ± 0.04 | 0.43 ± 0.06 | 0.28 ± 0.04 |
| Day 10 | 0.51 ± 0.06 | 0.40 ± 0.06 | 0.47 ± 0.05 | 0.31 ± 0.05^{a} |

Data represent mean acoustic startle response to a 120 dB pulse ± SEM.
^{a}indicates p ≦ 0.05 compared to same day mean startle response in vehicle group.

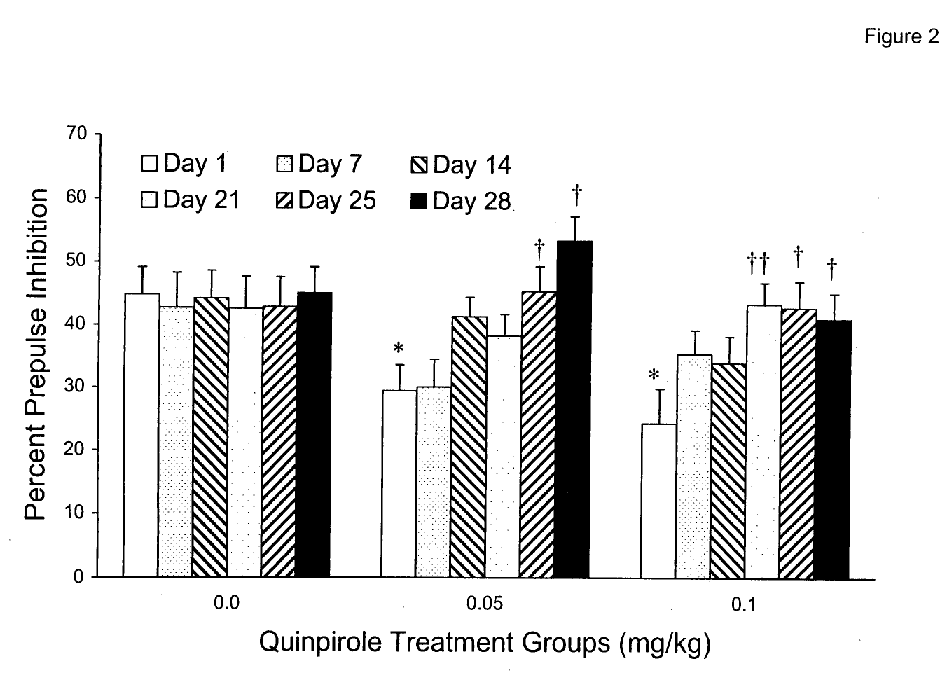

We also investigated the effect of longer drug exposure on PPI. Increasing doses of quinpirole reduced PPI on day 1 of treatment by 66% and 54% compared to vehicle treatment following 76 dB prepulses [F(2,65)=4.2, p ≦0.05] (FIG. 2). In fact, significant PPI disruption occurred on day 1 at all prepulse levels except after 73 dB prepulses in the low dose treatment group (Table 3).

TABLE 3
Effect of acute and 28 day repeated quinpirole administration on percent PPI
following a 73 or 82 dB prepulse.

| | Quinpirole Dose (mg/kg): | | | | | |
|---|---|---|---|---|---|---|
| Prepulse | 0.0 | 0.0 | 0.05 | 0.05 | 0.1 | 0.1 |
| Level | Day 1 | Day 28 | Day 1 | Day 28 | Day 1 | Day 28 |
| 73 dB | 29.1 ± 3.5 | 21.7 ± 5.5 | 19.6 ± 4.0 | 29.7 ± 3.7 | 9.0 ± 5.1^{a} | 20.5 ± 4.3 |
| 76 dB | 44.9 ± 4.3 | 45.1 ± 4.1 | 29.5 ± 4.2^{a} | 53.5 ± 3.9^{b} | 24.3 ± 5.6^{a} | 41.2 ± 4.1^{b} |
| 82 dB | 60.6 ± 3.7 | 63.6 ± 3.5 | 48.8 ± 3.2^{a} | 72.0 ± 3.2^{b} | 47.5 ± 3.5^{a} | 61.5 ± 3.9^{b} |

Data are expressed as percent PPI ± SEM.
^{a}indicates p ≦ 0.05 compared to day 1 vehicle PPI levels.
^{b}indicates p ≦ 0.05 compared to day 1 PPI within the treatment group.

PPI following 76 dB prepulses increased gradually with repeated quinpirole treatment, becoming significantly greater on treatment days 25 and 21 after treatment with 0.05 and 0.1 mg/kg quinpirole, respectively. Both quinpirole treatment groups displayed a full recovery of normal PPI levels by day 28, when PPI following all prepulse levels did not differ from that in the vehicle group [73 dB prepulse: F(2,64)=1.1, p=0.3; 76 dB prepulse: F(2,64)=0.5, p=0.6; 82 dB prepulse: F(2,64)=2.4, p=0.1], and was significantly higher than day 1 after both quinpirole doses following 76 and 82 dB prepulses [76 dB prepulse: F(5, 130)=5.6, p≦0.005; F(3,132)=2.9, p≦0.05; 82 dB prepulse: F(5, 130)=10.4, p≦0.005; F(5, 132)=4.9, p≦0.005] (FIG. 2 and Table 3). Neither acute nor 28 days of repeated quinpirole treatment affected mean startle responses to pulse trials (Table 4).

TABLE 4
Effect of 28 day repeated quinpirole administration on acoustic startle
response.

Quinpirole Dose (mg/kg):

| | 0.0 | 0.05 | 0.1 |
|---|---|---|---|
| Day 1 | 0.65 ± 0.07 | 0.69 ± 0.06 | 0.53 ± 0.6 |
| Day 7 | 0.64 ± 0.08 | 0.66 ± 0.07 | 0.54 ± 0.06 |
| Day 14 | 0.67 ± 0.07 | 0.69 ± 0.07 | 0.56 ± 0.06 |
| Day 21 | 0.76 ± 0.08 | 0.77 ± 0.06 | 0.70 ± 0.07 |
| Day 25 | 0.73 ± 0.09 | 0.76 ± 0.09 | 0.63 ± 0.07 |
| Day 28 | 0.65 ± 0.10 | 0.77 ± 0.10 | 0.61 ± 0.10 |

Data represent mean acoustic startle response to a 120 dB pulse ± SEM.

More recent studies demonstrated that the D2 receptor agonist ropinirole acts like quinpirole to attenuate PPI symptoms over time and when administered at the same low doses as quinpirole.

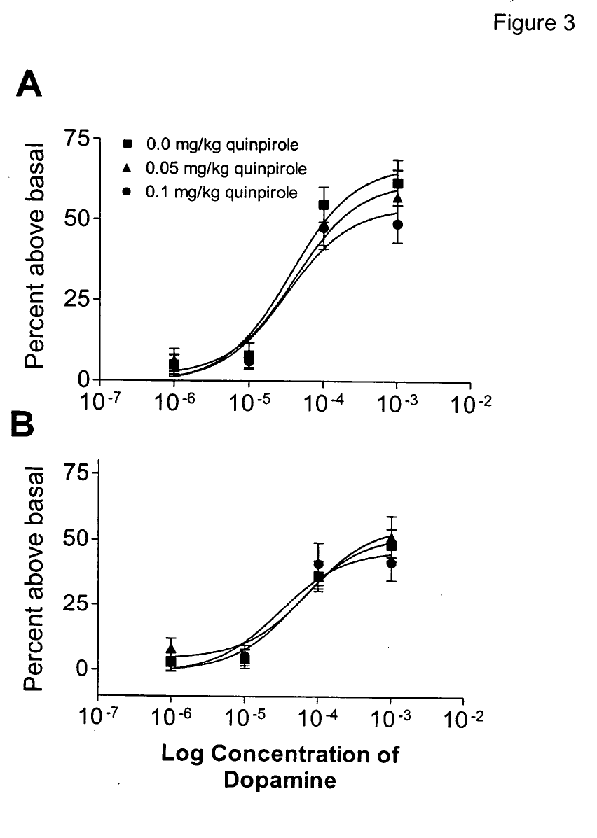

There was no significant effect of 10 day quinpirole treatment on the maximum efficacy of dopamine-mediated [³⁵S]GTPγS binding or on EC50 values in either the NAc core or shell (FIG. 3 and Table 5). For example, maximal dopamine efficacy in the NAc core after 0.05 and 0.1 mg/kg quinpirole treatment was 93% and 81% of vehicle group values, while EC50 values decreased by 12% after the low dose and increased by 13% after the intermediate quinpirole dose.

Similarly, 28 day quinpirole exposure had no effect on dopamine-mediated [³⁵S]GTPγS binding in the NAc core or shell (FIG. 4). Binding increased slightly in both regions after quinpirole treatment compared to vehicle treatment, but there was no significant effect of treatment on either basal (FIG. 4, inset) or dopamine-stimulated [³⁵S]GTPγS binding.

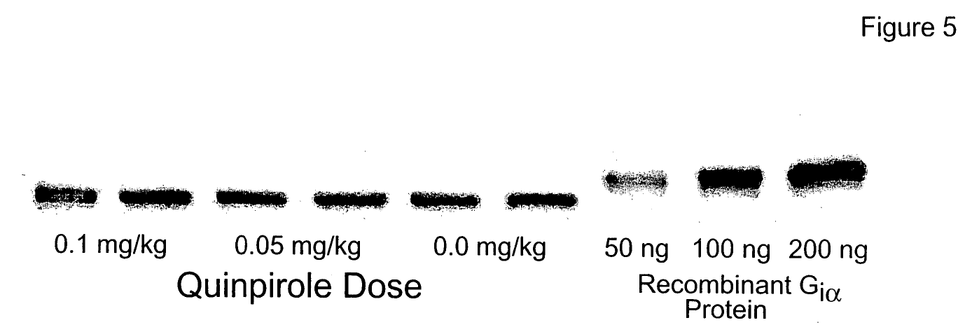

The effect of 28-day quinpirole treatment on NAc G proteins was assessed further by measuring levels of G protein immunoreactivity (FIG. 5). There were no significant differences between treatment groups in the amount of G_{1α} protein, nor were treatment effects detected in the levels of G_{sα} protein (Table 6).

TABLE 5
Effect of 10 day repeated quinpirole treatment on maximum efficacy
and EC50 of dopamine-stimulated [³⁵S]GTPγS binding in the NAc.

| | Quinpirole Dose (mg/kg): | | |
|---|---|---|---|
| | 0.0 | 0.05 | 0.1 |
| NAc Core: Maximal Efficacy | 67.0 ± 5.8% | 61.8 ± 6.9% | 54.0 ± 5.4% |
| (± SEM) | | | |
| NAc Core: EC50 (95% CI) | 35.6 μM | 31.2 μM | 40.4 μM |
| | (13, 98) | (9, 105) | (11, 146) |
| NAc Shell: Maximal Efficacy | 51.8 ± 5.9% | 55.7 ± 6.9% | 46.0 ± 6.4% |
| (± SEM) | | | |
| NAc Shell: EC50 (95% CI) | 51.5 μM | 73.0 μM | 29.3 μM |
| | (16, 169) | (21, 259) | (6, 149) |

TABLE 6
Effect of 28 day repeated quinpirole treatment on NAc G protein
immunoreactivity.

| | Quinpirole Dose (mg/kg): | | |
|---|---|---|---|
| | 0.0 | 0.05 | 0.1 |
| G_{iα} | 11.0 ± 1.1 | 11.4 ± 2.4 | 12.2 ± 1.3 |
| G_{sα}-42 kDa (major subunit) | 4.3 ± 0.8 | 4.2 ± 0.7 | 3.9 ± 1.0 |
| G_{sα}-47 kDa (minor subunit) | 2.3 ± 0.9 | 2.2 ± 0.8 | 2.4 ± 0.9 |
| G_{iα} | 11.0 ± 1.1 | 11.4 ± 2.4 | 12.2 ± 1.3 |

Standard curves constructed with known concentrations of recombinant G_{iα} or G_{sα} protein were used to quantify the relative immunoreactivity of NAc G proteins. Data are expressed in terms of ng of G protein/mg of total brain protein ± SEM for n = 7-8 per group.

The studies described above demonstrate that PPI was reduced after acute quinpirole administration, but gradually increased with repeated treatment. Quinpirole-induced PPI disruption was attenuated after 10 days of treatment at lower doses, but complete recovery was not apparent until the treatment period was extended to 25-28 days. Since chronic drug exposure can alter the dopamine system, we sought to characterize the effects of repeated quinpirole treatment on G proteins coupled to D2-like receptors in the NAc. [³⁵S]GTPγS binding and Western blot analysis revealed that repeated quinpirole treatment had no effect on NAc D2-like receptor-G protein function or G protein levels.

After 10 days of repeated treatment, quinpirole-mediated PPI disruption was significantly attenuated by 0.05 mg/kg treatment, but not at higher doses. Higher doses of quinpirole have been reported to significantly disrupt PPI in addition to reducing mean startle responses to pulse trials (Peng et al., Neuropsychopharmacology 3:211-218, 1990), as observed herein following repeated treatment with 0.3 mg/kg quinpirole. Extending the treatment period to 28 days, however, significantly increased PPI following both 0.05 and 0.1 mg/kg quinpirole doses. PPI recovery was complete after 3-4 weeks of treatment with both doses.

As noted above, we studied the functional coupling between receptors and G proteins in the NAc following repeated quinpirole treatment by [³⁵S]GTPγS binding analysis, which has been used to detect changes in receptor-effector coupling after chronic drug exposure. For example, chronic haloperidol administration increases D2 agonist-mediated [³⁵S]GTPγS binding to striatal membranes (Geurts et al., Eur. J. Pharmacol. 382:119-127, 1999). This assay can be used in rat brain sections to examine regional specificity of drug effects (He et al., 2000, supra), and a low concentration of Mg²⁺ in the assay buffer, which preferentially excludes Gₛ proteins (Waeber and Moskowitz, Mol. Pharmacol. 52:623-631, 1997), permits selective visualization of D2-like receptor activity by stimulation with dopamine, as confirmed previously (Culm et al., 2003, supra).

Utilizing a range of dopamine concentrations, we demonstrated that 10 day quinpirole exposure did not alter receptor-G protein coupling, having no significant effect on the maximum efficacy of dopamine nor on EC50 values (Table 5). Increasing dopamine concentrations produced a gradual increase in [³⁵S]GTPγS binding in all treatment groups, and dopamine-stimulated [³⁵S]GTPγS binding levels approached a plateau at 100 μM, as observed previously by He et al. (2000, supra). The striking similarities of the resulting sigmoidal curves of FIGS. 3A and 3B indicated that receptor-G protein coupling was not significantly altered after repeated quinpirole administration.

In an additional experiment, 100 μM dopamine-stimulated [³⁵S]GTPγS binding was assessed after a 28 day quinpirole treatment period. Basal levels of [³⁵S]GTPγS binding were slightly higher than in the assay of tissue from animals treated with quinpirole for only 10 days, which might have contributed to the relatively lower dopamine-stimulated [³⁵S]GTPγS percent binding above basal values in all groups of this experiment. No alteration of [³⁵S]GTPγS binding was observed after 28 days of quinpirole treatment, as neither basal nor dopamine-stimulated [³⁵S]GTPγS binding differed from the control group (FIG. 4).

NAc G proteins were further investigated after 28 day quinpirole treatment using quantitative Western blot analysis. Although repeated treatment with an indirect dopamine agonist reduced NAc G_{iα} and G_{oα} levels (Nestler et al., J. Neurochem. 55:1079-1082, 1990), the relatively low dose and selectivity of the D2-like dopamine agonist we utilized had no effect on NAc G_{iα} or G_{sα} protein levels (Table 6).

A number of embodiments of the invention have been described. Nevertheless, it will be understood that various modifications may be made without departing from the spirit and scope of the invention. Accordingly, other embodiments are within the scope of the following claims.

1. A method of treating a human patient who has experienced a psychotic symptom, the method comprising administering to the patient a composition comprising a therapeutically effective amount of a full agonist of a dopamine D2-like receptor.

2. The method of claim 1, wherein the psychotic symptom comprises a hallucination or delusion.

3. The method of claim 1, wherein the patient has been diagnosed as having a neuropsychiatric disorder.

4. The method of claim 3, wherein the neuropsychiatric disorder is schizophrenia, obsessive compulsive disorder or Tourette's syndrome.

5. The method of claim 1, wherein the full agonist is ropinirole, quinpirole, pramipexole, or apomorphine.

6. The method of claim 1, further comprising a step of identifying a patient who has experienced a psychotic symptom, the step of identifying the patient being carried out before the step of administering the composition.

7. The method of claim 1, wherein the amount of the full agonist in the composition is such that, upon repeated administration of the composition, the patient realizes one or more of the following benefits:
(a) the patient does not experience an external effect of the drug;
(b) the patient does not experience an extrapyramidal symptom (EPS) or experiences a tolerable level of an EPS; or
(c) upon cessation of treatment, the patient's psychotic symptom does not return as soon as expected.

8. The method of claim 1, wherein the composition is administered at least once a day for at least seven days.

9. The method of claim 1, wherein the composition is administered at least once a day for at least 30 days.

10. The method of claim 1, wherein the patient receives less than 0.4 mg/kg/day of the full agonist.

11. The method of claim 1, wherein the patient receives less than 0.04 mg/kg/day of the full agonist.

12. The method of claim 1, wherein the full agonist selectively activates a dopamine D2 receptor.

13. The method of claim 1, wherein the full agonist selectively activates a dopamine D3 or D4 receptor.

14. A method of treating a patient who has experienced a psychotic symptom, the method comprising administering to the patient a therapeutically effective amount of an antipsychotic agent, the agent being identified by a process comprising
(a) administering a test compound to a test subject; and
(b) determining whether the test compound attenuates a startle response, wherein a test compound that attenuates the startle response is a candidate antipsychotic agent.

15. The method of claim 14, wherein the step of determining whether the test compound attenuates a startle response comprises comparing the extent of prepulse inhibition (PPI) in a test subject or a population of test subjects exposed to the test compound with the extent of PPI in a test subject or a population of test subjects who have not been exposed to the test compound, wherein a test compound that increases the extent of PPI is a candidate antipsychotic agent.

16. The method of claim 14, further comprising administering the candidate antipsychotic agent to a patient who has experienced a psychotic symptom, wherein a candidate antipsychotic agent that improves the psychotic symptom is an antipsychotic agent.

18. The method of claim 14, wherein the test subject is a rodent.

19. The method of claim 14, wherein the administering and determining steps are repeated at least two times.

20. The method of claim 14, wherein the test compound is a small inorganic molecule, an antibody or a fragment or variant thereof, or a nucleic acid that inhibits gene expression.

21. A pharmaceutical composition comprising
(a) a therapeutically effective amount of a first agent, wherein the first agent is a full agonist of a dopamine D2-like receptor, and
(b) a therapeutically effective amount of a second agent, wherein the second agent is an antipsychotic or antidepressant other than a full agonist of a dopamine D2-like receptor.

22. The pharmaceutical composition of claim 21, wherein the second agent is an atypical antipsychotic.

23. The pharmaceutical composition of claim 22, wherein the atypical antipsychotic is aripiprazole, risperidone, clozapine, olanzapine, quetiapine, or ziprasidone.
